(19) United States
(12) Patent Application Publication
(10) Pub. No.: US 2022/0288318 A1
(43) Pub. Date: Sep. 15, 2022

(54) ACTIVATABLE INJECTION DEVICE FOR DRUG DELIVERY

(71) Applicant: AMGEN INC., Thousand Oaks, CA (US)

(72) Inventors: Christian Plambech, Soeborg (DK); Bjarke Lykke Ludvig Svendsen, Slagelse (DK)

(21) Appl. No.: 17/637,520

(22) PCT Filed: Aug. 25, 2020
(86) PCT No.: PCT/US20/47815

§ 371 Date: Feb. 23, 2022

Related U.S. Application Data
(60) Provisional application No. 62/895,041, filed on Sep. 3, 2019.

(51) Int. Cl. A61M 5/20; A61M 5/32

(52) U.S. Cl. CPC A61M 5/2033; A61M 5/321; A61M 2005/2013

(57) ABSTRACT

Injection devices for drug delivery are disclosed. An injection device may include a housing having an opening, a drug storage container including a delivery member with an insertion end configured to extend at least partially through the opening, and a plunger. A drive mechanism may be included for expelling a drug from the drug storage container through the delivery member. The drive mechanism may be activated by a guard member moveably disposed in the opening in the housing, an activator member moveable independent of the guard member and which may be moveably disposed in the opening of the housing, and/or a portion of the housing that is moveable relative to another portion of the housing.

### CROSS-REFERENCE TO RELATED APPLICATIONS

Priority is claimed to U.S. Provisional Patent Application No. 62/895,041, filed Sep. 3, 2019, and the entire contents thereof are incorporated herein by reference.

### FIELD OF THE DISCLOSURE

This disclosure generally relates to injectors for drug delivery and, more particularly, the activation of such devices.

### BACKGROUND

Patients receive drugs to treat a variety of medical conditions. While certain drugs are administered via oral, topical, or inhalation routes, other drugs are administered via an injection. An injection involves piercing the patient's skin with a delivery member such as a needle or cannula and forcing a drug through the delivery member into the patient.

Conventionally, syringes have been used to administer injectable drugs. Use of a syringe requires manually inserting a needle into the skin and then manually pushing a plunger to force a drug out through the needle into the patient. Performing these steps requires dexterity and skill, which makes self-administration with a syringe challenging for certain individuals. Syringes also involve a risk of accidental needle sticks because the needle may be exposed prior to and after the injection.

To facilitate self-administration, certain injectors automate various aspects of the injection process and include a drive mechanism pursuant to these ends. It is generally desirable for the activation of the drive mechanism to be intuitive for the patient and involve relatively few steps. It is also desirable for any activation mechanism to be able to interact with the drive mechanism without adding undue complexity or cost to device. Achieving these objectives and others, such as providing for an elongate, pen-like shape in the case of an autoinjector, presents various design and manufacturing challenges.

The present disclosure sets forth injection devices embodying advantageous alternatives to existing injection devices, and that may address one or more of the challenges or needs mentioned herein, as well as provide other benefits and advantages.

### SUMMARY

One aspect of the present disclosure provides an injection device including a housing, a drug storage container, a plunger, a biasing member, and a guard member. The housing may have an opening, and the drug storage container may include a delivery member having an insertion end configured to extend at least partially through the opening in the housing. The biasing member may be operably coupled to the plunger and initially retained in an energized state. Releasing the biasing member may cause the biasing member to drive the plunger to expel a drug from the drug storage container through the delivery member. The guard member may have a skin-contacting portion and an activator portion. Further, the guard member may be moveable relative to the housing and have an extended position wherein the guard member extends at least partially through the opening in the housing and a retracted position wherein the guard member is positioned away from the extended position toward the housing. Moving the guard member from the extended position to the retracted position may cause the activator portion to release the biasing member to allow the biasing member to drive the plunger to expel the drug from the drug storage container.

Another aspect of the present disclosure provides an injection device including a housing, a drug storage container, a plunger, a drive mechanism, a guard member, and an activator member. The housing may have an opening, and the drug storage container may include a delivery member having an insertion end configured to extend at least partially through the opening in the housing. The drive mechanism may be activatable to expel a drug from the drug storage container through the delivery member. The guard member may be moveable relative to the housing and have an extended position wherein the guard member extends at least partially through the opening in the housing and a retracted position wherein the guard member is positioned away from the extended position toward the housing. The activator member may be moveable relative to the housing independent of movement of the guard member.

A further aspect of the present disclosure provides an injection device including a distal housing, a drug storage container, a plunger, a drive mechanism, and a proximal housing. The distal housing may have an opening, and the drug storage container may include a delivery member having an insertion end configured to extend at least partially through the opening in the distal housing. The drive mechanism may be activatable to drive the plunger in a distal direction to expel a drug from the drug storage container through the delivery member. The proximal housing may be operably coupled to the drive mechanism and moveable relative to the distal housing such that moving the proximal housing in the distal direction activates the drive mechanism.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 is a schematic cross-sectional representation of an injection device according to an embodiment of the present disclosure.

FIG. 2 is a perspective view of a guard member according to an embodiment of the present disclosure.

FIGS. 3A-3D illustrate an activation sequence of an embodiment of an injection device incorporating the guard member of FIG. 2.

FIGS. 4A and 4B depict an activation sequence of an injection device according to another embodiment of the present disclosure.

FIGS. 5A and 5B are perspective views of a guard member according to another embodiment of the present disclosure.

FIG. 6 is a schematic cross-sectional representation of an injection device according to another embodiment of the present disclosure.

FIG. 7 is a perspective view of a guard member according to another embodiment of the present disclosure.

FIGS. 8A-8D illustrate an activation sequence of an embodiment of an injection device incorporating the guard member of FIG. 7.

FIGS. 9A-9C depict an activation sequence of an injection device according to another embodiment of the present disclosure.

FIGS. 10A-10C illustrate an activation sequence of an injection device according to another embodiment of the present disclosure.

FIGS. 11A-11F depict an activation sequence of an injection device according to another embodiment of the present disclosure.

FIGS. 12A-12C illustrate an activation sequence of an injection device according to another embodiment of the present disclosure.

FIG. 13 illustrate an injection device according to another embodiment of the present disclosure.

FIG. 14 is a schematic cross-sectional representation of an injection device according to an embodiment of the present disclosure.

FIGS. 15A-15D depict an activation sequence of an injection device according to another embodiment of the present disclosure.

FIG. 16 is a schematic cross-sectional representation of an injection device according to an embodiment of the present disclosure.

FIGS. 17A-17C illustrate an activation sequence of an injection device according to another embodiment of the present disclosure.

FIGS. 18A-18C depict an activation sequence of an injection device according to another embodiment of the present disclosure.

FIG. 19 is a schematic cross-sectional representation of an injection device according to an embodiment of the present disclosure.

FIGS. 20A-20D depict an activation sequence of an injection device according to another embodiment of the present disclosure.

### DETAILED DESCRIPTION

The present disclosure generally relates to injection devices which can be safely and reliably activated by a user for administering a drug, or in the case where a patient is the user, self-administering a drug. Certain embodiments herein allow the user to activate or unlock a drive mechanism by pushing the injection device against the injection site. Thus, after positioning the injection device at the injection site, the user is not required to change his or her grip and/or employ a second hand in order to activate or unlock the drive mechanism. This streamlines use of the device and reduces the likelihood of an erroneous or suboptimal injection.

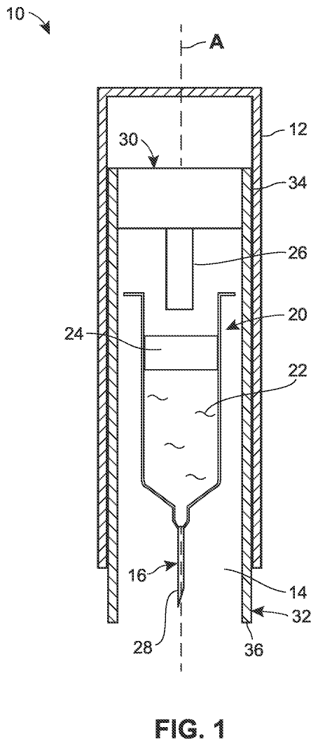

FIG. 1 is a schematic representation of an injection device 10 according to an embodiment of the present disclosure. The injection device 10 may be configured as a single-use, disposable injector or a multiple-use reusable injector. The injection device 10 may be configured to inject any suitable drug or combination of drugs. The injection device 10 may be intended for self-administration but also may be used by a caregiver or a formally trained healthcare provider (e.g., a doctor or nurse) to administer an injection. In some embodiments, the injection device 10 may be configured as an autoinjector or a pen-type injector and as such may be held in the users hand over the duration of drug delivery. In embodiments where drug delivery may be delayed or take several minutes or hours, the injection device 10 may be configured as an on-body injector (e.g., a patch injector) which can be releasably attached to the patient's skin via, for example, an adhesive.

As depicted in FIG. 1, the injection device 10 includes an outer casing or housing 12. In some embodiments, the housing 12 may be sized and dimensioned to enable a person to grasp the injector 10 in a single hand. The housing 12 may have a generally elongate shape, such as a cylindrical shape, extending along a longitudinal axis A between a proximal end and a distal end. An opening 14 may be formed in the distal end to permit an insertion end 28 of a delivery member 16 to extend outside of the housing 12. A transparent or semi-transparent inspection window may be positioned in a wall of the housing 12 to permit a user to view component(s) inside the injection device 10 including a drug storage container 20. Viewing the drug storage container 20 through the window may allow a user to confirm that drug delivery is in progress and/or complete. A removable cap (not illustrated) may cover the opening 14 prior to use of the injection device 10, and, in certain embodiments, may be coupled to and assist with removing a sterile barrier (e.g., a rigid needle shield) mounted on the insertion end 28 of the delivery member 16.

The drug storage container 20 is disposed within an interior space of the housing 12 and is configured to contain a drug 22. The drug storage container 20 may be pre-filled and shipped by, for example, a manufacturer, or, alternatively, filled by a user prior to use of the injection device 10. The housing 12 may be pre-loaded with the drug storage container 20 by, for example, a manufacturer, or alternatively, loaded with the drug storage container 20 by a user prior to use of the injection device 10. The drug storage container 20 may include a rigid wall defining an internal bore or reservoir. The wall may be made of glass or plastic. In some embodiments, the drug storage container 20 may have a flexible or deformable wall and take the form of a collapsible pouch or bladder. In the illustrated embodiment, a stopper 24 is moveably disposed in the drug storage container 20 such that it can move in a distal direction along the longitudinal axis A between proximal end and a distal end of the drug storage container 20. The stopper 24 may be constructed of rubber or any other suitable material. The stopper 24 may slidably and sealingly contact an interior surface of the drug storage container 20 such that the drug is prevented or inhibited from leaking past the stopper 24 when the stopper 24 is in motion. Distal movement of the stopper 24 expels the drug 22 from the drug storage container 20 through the delivery member 16 as the stopper 24 is driven in the distal direction. The proximal end of the drug storage container 20 may be open to allow a plunger 26 to extend into the drug storage container 20 and push the stopper 24 in the distal direction. In the present embodiment, the plunger 26 and the stopper 24 are initially spaced from each other and the plunger 26 impacts the stopper 24 during operation of the injection device 10. In alternative embodiments, the stopper 24 and the plunger 26 may be coupled to each other, e.g., via a threaded coupling, such that they move together jointly from the start of movement of the plunger 26. In embodiments where the drug storage container 20 takes the form of a collapsible pouch or bladder, the stopper 24 may be omitted and the plunger 26 may press on an exterior surface of a wall of the drug storage container 20 in order to deform the wall and reduce an interior volume of the drug storage container 20, thereby expelling the drug 22.

The delivery member 16 is connected or operable to be connected in fluid communication with the reservoir of the drug storage container 20. A distal end of the delivery member 16 may define an insertion end 28 of the delivery member 16. The insertion end 28 may include a sharpened tip of other pointed geometry allowing the insertion end 28 to pierce the patient's skin and/or subcutaneous tissue during insertion of the delivery member 16. The delivery member 16 may be hollow and have an interior passageway. One or more openings may be formed in the insertion end 28 to allow drug to flow out of the delivery member 16 into the patient. In some embodiments, the delivery member 16 may be defined by a single structure such as a rigid needle or a flexible cannula; whereas, in other embodiments, the delivery member 16 may be defined by multiple interdependent structures. With regard to the latter, the delivery member 16 may include in certain embodiments a rigid metal needle and a flexible plastic cannula, where the needle is used to insert the cannula into the patient and thereafter partially or fully retracts from the cannula leaving the cannula within the patient for subcutaneous delivery. Such an arrangement may be desirable from a safety and/or comfort perspective, particularly if the cannula is to be left within the patient's body for a significant period of time (e.g., minute(s), hour(s), day(s), etc.).

In the embodiment illustrated in FIG. 1, the drug storage container 20 is a pre-filled syringe and has a staked, hollow metal needle defining the delivery member 16. Here, the needle is fixed relative to the wall of the drug storage container 20 and is in permanent fluid communication with the reservoir of the drug storage container 20. In other embodiments, the drug storage container 20 may be a needle-less cartridge, and, as such, initially may not be in fluid communication with the delivery member 16. In such embodiments, the drug storage container 20 may move toward a proximal end of the delivery member 16, or vice versa, during operation of the injection device 10 such that the proximal end of the delivery member 16 penetrates through a septum covering an opening in the drug storage container 20, thereby establishing fluid communication with the reservoir of the drug storage container 20.

In some embodiments, the drug storage container 20 may be fixed to the housing 12 such that the drug storage container 20 does not move relative to the housing 12 after being installed within the housing 12. In such embodiments, including the one depicted in FIG. 1, the insertion end 28 of the delivery member 16 may extend through the opening 14 in the housing 12 in both an initial or storage state and in a post-delivery state. In alternative embodiments, the drug storage container 20 may be moveably coupled to the housing 12 such that the drug storage container 20 is able to move relative to the housing 12 during operation of the injection device 10. In certain such embodiments, the insertion end 28 of the delivery member 16 may be retracted within the opening 14 in the housing 12 in the initial or storage state. Subsequently, during operation of the injection device 10, the insertion end 28 of the delivery member 16 may be deployed through the opening 14 in the housing 12 for insertion into the patient. In some embodiments, this motion may be the result of the drug storage container 20 being driven in the distal direction relative to the housing 12

Still referring to FIG. 1, the injection device 10 further includes a drive mechanism 30 mounted within the housing 12. The drive mechanism 30 may be configured to store energy and, upon or in response to activation of the drive mechanism 30 by the user, release or output that energy to drive the plunger 26 to expel the drug 22 from the drug storage container 20 through the delivery member 16 into the patient. For example, the drive mechanism 30 may be configured to store mechanical, electrical, and/or chemical potential energy and, upon activation of the drive mechanism 30, convert that potential energy into kinetic energy or motion of the plunger 26. The drive mechanism 30 may be disposed partially or entirely within the housing 12. The drive mechanism 30 may be directly coupled to the plunger 26 or coupled to the plunger 26 via an intervening mechanical or electromechanical linkage.

In some embodiments, the drive mechanism 30 may be powered by a biasing member, such as a spring, which is initially retained in an energized state. In the energized state, the biasing member may be compressed, tensioned, torqued (e.g., twisted or wound), or any combination thereof, depending on the construction of biasing member. In the energized state, the biasing member may exert a biasing force on the plunger 26 but is prevented from moving the plunger 26 by a retaining arrangement, as described below. When released, the biasing member may return to its natural length or shape, and as a consequence, drive the plunger 26 to expel the drug 22 from the drug storage container 20. In some embodiments, the biasing member may be a linear biasing member configured to exert a biasing force causing linear motion; whereas, in other embodiments, the biasing member may be a rotational biasing member configured to exert a biasing force causing rotational motion. In embodiments where the biasing member includes a spring, the spring may be any one or combination of a helical compression spring, a helical extension spring, a helical torsion spring, a spiral torsion spring, or any other suitable spring. In addition to or as an alternative to the biasing member, the drive mechanism 30 may include any one or combination of: an electromechanical arrangement including an electric motor and/or solenoid and a drive train or transmission coupled to the plunger 26; or an arrangement that generates or releases a pressurized gas or fluid to propel the plunger 26 or which acts directly on the stopper 24 to move stopper 24 through the drug storage container 20 to expel the drug 22 from therein. In embodiments where the drug storage container 20 and/or the delivery member 16 is moveable relative to the housing 12, the drive mechanism 30 may, upon activation, drive the drug storage container 20 and/or the delivery member 16 in the distal direction so as to cause the insertion end 28 of the delivery member 16 to be inserted into the patient. Thus, in certain embodiments, the drive mechanism 30 may provide the motive force needed for both inserting the delivery member 16 into the patient and expelling the drug 22 from the drug storage container 20.

With continued reference to FIG. 1, the injection device 10 includes a guard member 32 for preventing contact with the insertion end 28 of a delivery member 16 when the injection device 10 is not being used to administer an injection. The guard member 32 may have a proximal end received within the housing 12, and configured to move relative to the housing 12 between an extended position wherein a distal end of the guard member 32 extends through the opening 14 in the housing 12 and a retracted position wherein the distal end of the guard member 32 is retracted, fully or partially, into the opening 14 in the housing 12. In at least the extended position, the guard member 32 may extend beyond and surround the insertion end 28 of the delivery member 16. In some embodiments, moving the guard member 32 toward the retracted position may expose the insertion end 28 of the delivery member 16. In some embodiments, the guard member 32 may be coupled to the housing 12 via, for example, a pin-and-slot arrangement or similar such that the guard member 32 is able to translate in a linear direction relative to the housing 12 but is prevented from rotating relative to the housing 12.

The proximal and distal ends of the guard member 32 may include, respectively, an activator portion 34 and a skin-contacting portion 36. In some embodiments, the activator portion 34 and the skin-contacting portion 36 may be integrally formed to define a single, monolithic structure. Said another way, the activator portion 34 and the skin-contacting portion 36 may be formed in one piece. In other embodiments, the activator portion 34 and the skin-contacting portion 36 may be physically separate structures that are fixedly attached to each other such that they are immovable relative to each other and/or move jointly when in motion. At least the skin-contacting portion 36 of guard member 32 may have a tubular or cylindrical shape and, in some embodiments, may be centered about the longitudinal axis A of the injection device 10. Moving the guard member 32 from the extended position to the retracted position may be accomplished by pressing the skin-contacting portion 36 against the patient's skin at the injection site. In embodiments where the delivery member 16 protrudes from the opening 14 in the housing 12 in the initial or storage state, this motion may result in the insertion end 28 of the delivery member 16 being inserted into the patient's skin.

In some embodiments, the guard member 32 may be biased towards the extended position by a biasing member such as a spring. A user may overcome a biasing force provided by this biasing member by pressing the guard member 32 against the injection site. When the injection is complete and the injection device 10 is lifted off of the injection site, the biasing member may return the guard member 32 to the extended position, thereby covering the insertion end 28 of the deliver member 16. In some embodiments, the injection device 10 may include a lockout mechanism for locking the guard member 32 in the extended position after the guard member 32 has moved from the retracted position to the extended position in order to prevent re-use of the injection device 10 and/or to reduce the likelihood of unintended needle pokes.

In some embodiments, the guard member 32 may be configured to interact with the drive mechanism 30 when the guard member 32 moves from the extended position to the retracted position. This interaction may cause the drive mechanism 30 to output the energy necessary for driving the plunger 26 to expel the drug 22 from the drug storage container 20 and/or insert the insertion end 28 of the delivery member 16 into the patient's skin. The interaction between the guard member 32 and the drive mechanism 30 may be achieved by directly coupling the guard member 32 to the drive mechanism 30 or indirectly coupling the guard member 32 to the drive mechanism 30 via, for example, a mechanical or electromechanical linkage. In embodiments where the drive mechanism 30 includes a biasing member such as a spring, movement of the guard member 32 from the extended position to the retracted position may release the biasing member from an energized state to allow the biasing member to drive the plunger 26 to expel the drug 22 from the drug storage container 20. Additionally or alternatively, the guard member 32 may be configured to retain the biasing member in the energized state when the guard member 32 is arranged in the extended position. In some embodiments, the guard member 32 may retain the biasing member via direct contact with the biasing member, the plunger 26, and/or an element fixedly attached to the biasing member or plunger 26. In embodiments where the biasing member exerts a biasing torque, the guard member 32 may retain the biasing member by preventing rotation of the biasing member and/or a rotational element which is biased to rotate by the biasing member such as the power sleeve 555 described below. In further embodiments where the guard member 32 is used to retain the biasing member in the energized state, the activator portion 34 of the guard member 32 may be deformable and may experience deformation as a consequence of the guard member 32 moving from the extended position to the retraction position. This deformation causes the activator portion 34 to release the biasing member or an element biased by the biasing member, thereby allowing the biasing member to de-energize and drive the plunger 26 to expel the drug 22 from the drug storage container 20. In certain such embodiments, the activator portion 34 of the guard member 32 may include one or more deformable arms which deflect radially outwardly to release the biasing member when the guard member 32 moves from the extended position to the retracted position. The deflection of the arms may, in some embodiments, be caused by the guard member 32 pressing against an angled ledge or lip formed on the inner surface of the housing 12, which creates a torque bending the deformable arms outwardly. In a variation on this embodiment, one or more resilient arms which are separate from the guard member 32 may be held by direct contact with the activator portion 34 of the guard member 32 in a first or compressed position where the resilient arms prevent movement of the biasing member, the plunger 26, and/or an element fixedly attached to the biasing member or plunger 26. When the guard member 32 moves from the extended position to the retracted position, the activator portion 34 of the guard member 32 may move out of contact with the resilient arms, thereby freeing the resilient arms to return to their original or natural shape and thus move to a second position. In the second position, the resilient arms may no longer restrain the biasing member and thus allow the biasing member to de-energize and drive the plunger 26 to expel the drug 22 from the drug storage container 20.

In embodiments where the drive mechanism 30 includes an energy source that is electromechanical arrangement including an electric motor and/or solenoid and a drive train or transmission coupled to the plunger 46 or an arrangement that generates or releases a pressurized gas or fluid to propel the plunger 26 or which acts directly on the stopper 424, the guard member 32 may directly act on (i.e., directly contact and exert a force on) the drive mechanism 30 to activate the drive mechanism 30 when the guard member 32 moves from the extended position to the retracted position.

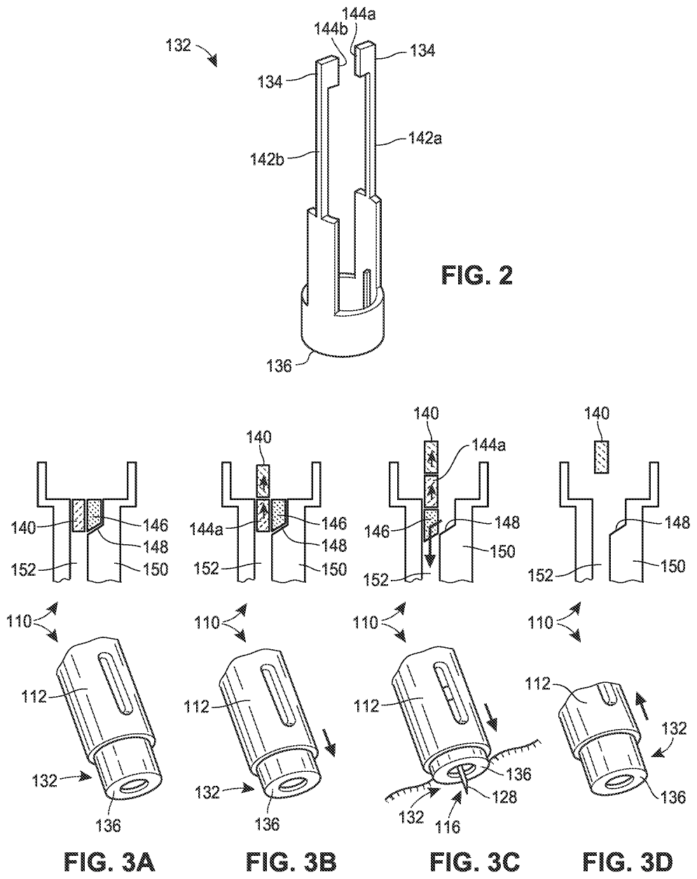

Referring now to FIGS. 2-3D, illustrated is an embodiment of a guard member which can be implemented in an injection device including, for example, the injection device 10 illustrated in FIG. 1. Elements in FIGS. 2-3D which are similar in function and/or structure to elements in FIG. 1 are designated by the same reference numeral, incremented by 100 relative to their counterparts in FIG. 1. The guard member 132 here is configured to interact with a retaining member 140, which is a structure that is separate from the guard member 132, as a consequence of the guard member 132 moving from the extended position to the retracted position. This interaction releases a biasing member included in the drive mechanism 30 such that the biasing member is permitted to drive the plunger 26 to expel the drug 22 from the drug storage container 20. In some embodiments, the retaining member 140 may be part of the drive mechanism 30 depicted in FIG. 1.

FIG. 2 illustrates that the skin-contacting portion 136 of the guard member 132 may have a tubular or cylindrical shape. Two longitudinally extending arms 142a and 142b extend away from the skin-contacting portion 136 in the proximal direction. The skin-contacting portion 136 may be integrally formed with longitudinally extending arms 142a and 142b to define a single, monolithic structure. Said another way, the skin-contacting portion 136 and the longitudinally extending arms 142a and 142b may be formed in one piece. The longitudinally extending arms 142a and 142b may be parallel or substantially parallel to the longitudinal axis of the injection device 10 and arranged so as to not cover one or more windows formed in the housing 12 of the injection device 10.

One or both of the proximal ends of the longitudinally extending arms 142a and 142b may define the activator portion 134 of the guard member 132. In the embodiment illustrated in FIG. 2, the proximal ends of the longitudinally extending arms 142a and 142b include, respectively, walls 144a and 144b, each of which extends inwardly away from a remainder of the longitudinally extending arm. The walls 144a and 144b may be perpendicular or substantially perpendicular, or otherwise non-parallel, to the longitudinal axis A of the injection device 110.

Referring to FIGS. 3A-3D, a sequence in which the guard member 132 is used to release a biasing member of the drive mechanism of the injection device 10 will now be explained. FIG. 3A illustrates an initial state of the injection device 110 prior to activation. Here, the biasing member of the drive mechanism is retained in an energized state by the retaining member 140 arranged in a first position. In the energized state, the biasing member may exert a force biasing the plunger in the distal direction. As a consequence, a flange 146 extending radially outwardly from the plunger is pushed downwardly against an angled camming surface 148 of a sleeve 150 that is fixedly attached to the housing 112. The biasing force urges the flange 146 to slide down the camming surface 148 towards a longitudinally extending slot 152 formed in the sleeve 150. Initially, the flange 146 is prevented from sliding down the camming surface 148 by the retaining member 140, which abuts the flange 146 when the retaining member 140 is arranged in the first position. FIG. 3A shows that no portion of the guard member 132 is in contact with the retaining member 140 in the initial state; however, in other embodiments, the activator portion 134 of the guard member 132 may be in direct contact with the retaining member 140 in the initial state. It is noted that the flange 146 may be integrally formed with the plunger or may be a separate component that is fixedly attached to the plunger such that the flange 146 moves jointly together with the plunger.

FIG. 3B illustrates a state of the injection device 110 after the skin-contacting portion 136 of the guard member 132 has been pressed against the patient's skin and partially retracted within the opening 114 of the housing 112. Here, the wall 144a of the activator portion 134 of the guard member 132 directly contacts and pushes the retaining member 140 in the proximal direction. As a consequence, the retaining member 140 is moved from the first position to a second position where the retaining member 140 no longer restrains the flange 146 from sliding down the camming surface 148. As a result of this action, the wall 144a momentarily assumes the first position of the retaining member 140 and thus momentarily retains the biasing member in the energized state by abutting against and preventing the flange 146 from sliding down the camming surface 148. Though not illustrated, the wall 144b of the activator portion 134 of the guard member 132 may perform a similar action on the other side of the plunger.

Upon further retraction of the guard member 132, the wall 144a may slide out of contact with the flange 146, as shown in FIG. 3C. With nothing to retain it, the flange 146 may slide down the camming surface 148 and then into the longitudinally extending slot 152 under the biasing force of the biasing member. In some embodiments, the plunger may rotate relative to the sleeve 150 as the flange 146 slides down the camming surface 148. When the flange 146 is within the longitudinally extending slot 152, the biasing member may continue to de-energize, thereby driving the plunger in the distal direction to expel the drug 22 from the drug storage container 20. Once a dose of the drug 22 has been delivered to the patient, the injection device 110 may be lifted off of the patient's skin and the guard member 132 may return to the extended position under the force of a needle guard biasing member, as shown in FIG. 3D.

In alternative embodiments, the activator portion 134 of the guard member 132, instead of the sleeve 150, may include the camming surface. In such embodiments, the flange 146 may initially rest against a non-angled stop surface included in the sleeve 150 or other member such that the flange 146 is not biased to rotate in the initial state. When the guard member 132 moves from the extended position to the retracted position, the camming surface on the activator portion 134 of the guard member 132 may directly contact and push against the flange 146, thereby causing the flange 146 to rotate to a position where it is aligned with the longitudinally extending slot 152 in the sleeve 150 or another position where it is no longer restrained in the distal direction by the stop surface of the sleeve 150.

In further alternative embodiments, a non-physical interaction between the activator portion 134 of the guard member 132 and the retaining member 140 may move the retaining member 140 from the first position to the second position. In certain such embodiments, the activator portion 134 of the guard member 132 and the retaining member 140 may be magnetically repelled from or magnetically attracted to each other. The force associated with the magnetic repulsion or attraction may increase as the guard member 132 moves from the extended position to the retracted position. As a consequence, the magnetic repulsion or attraction between the activator portion 134 of the guard member 132 and the retaining member 140 may move the retaining member 140 from the first position to the second position, thereby freeing the biasing member to drive the plunger 126 in the distal direction to expel the drug from the drug storage container.

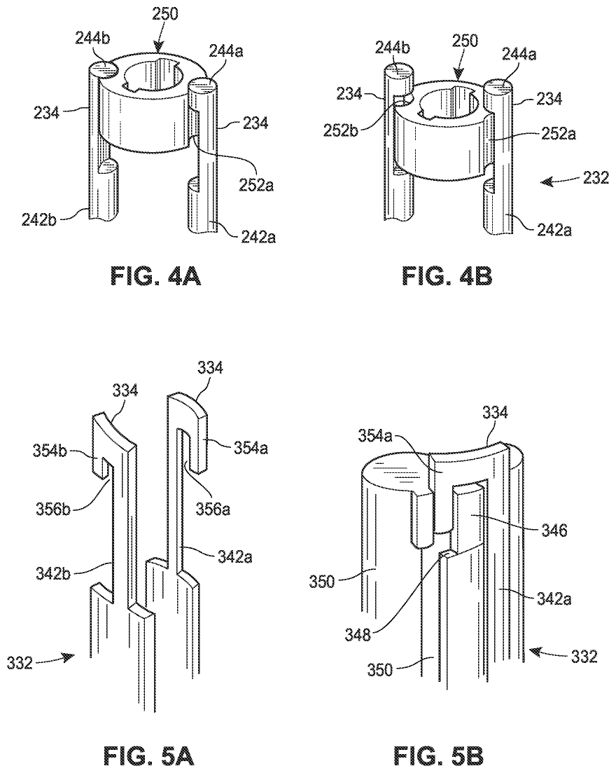

While the foregoing embodiment utilizes the retaining member 140 for initially retaining the biasing member in the energized state, other embodiments may omit the retaining member 140 and instead utilize the guard member for initially retaining the biasing member in the energized state. FIGS. 4A and 4B illustrate such an embodiment. Elements in FIGS. 4A and 4B which are similar in function and/or structure to elements in FIGS. 2-3D are designated by the same reference numeral, incremented by 100 relative to their counterparts in FIGS. 2-3D. In the embodiment in FIGS. 4A and 4B, the injection device may include a releaser member 250. The releaser member 250 may have a tubular or cylindrical shape and may be disposed around the plunger (not shown in FIGS. 4A and 4B). The releaser member 250 may be biased to rotate under a force exerted by the biasing member. When the releaser member 250 is free to rotate, it may rotate from a first rotational position to a second rotational position. In the first rotational position, the releaser member 250 may directly or indirectly engage the plunger to prevent the plunger from moving in the distal direction; in the second rotational position, the releaser member 250 may release the plunger to allow the plunger to move the distal direction under the biasing force of the biasing member to expel the drug 22 from the drug storage container 20.

FIG. 4A depicts the guard member 232 member in an extended position and the releaser member 250 in the first rotational position. Here, the walls 244a and 244b the activator portion 234 of the guard member 232 are received within and directly contact respective grooves 252a and 252b formed in the outer peripheral surface of the releaser member 250. As a result of this mating engagement, the activator portion 234 of the guard member 232 prevents the releaser member 250 from rotating and therefore retains the biasing member in the energized state. When the guard member 232 is pressed against the patient's skin and retracts into the opening 14 of the housing 12, the walls 244a and 244b may slide out of their respective grooves 252a and 252b, as shown in FIG. 4B. As a consequence, the releaser member 250 may be free to rotate from the first rotational position to the second rotation position under the biasing force of the biasing member. This motion, in turn, may cause grooves formed in an inner surface of the releaser member 250 to align with a flange extending from the plunger, thereby allowing the plunger to move in the distal direction under the biasing force of the biasing member to expel the drug from the drug storage container.

Turning to FIGS. 5A and 5B, the embodiment depicted here is generally a hybrid of the embodiment in FIGS. 2-3D and the embodiment in FIGS. 4A and 4B. The injection device in FIGS. 5A and 5B may include a flange 346, camming surface 348, sleeve 350, and longitudinally extending slot 352 similar in function and structure to, respectively, the flange 146, camming surface 148, sleeve 150, and longitudinally extending slot 152 of the embodiment in FIGS. 3A-3D. Similar to the embodiment in FIGS. 4A and 4B, the guard member 332 in the embodiment in FIGS. 5A and 5B is configured to retain the biasing member in the energized state when the guard member 332 is in the extended position (FIG. 5B). To achieve this, the activation portion 334 of the guard member 332 generally takes the form of a u-shaped hook. More particularly, and with reference to FIG. 5A, the proximal ends of the longitudinally extending arms 342a and 342b may include, respectively, walls 354a and 354b, each of which extends laterally away from a remainder of the longitudinally extending arm in a generally circumferential direction relative to the longitudinal axis A of the injection device. Cut-outs or grooves 356a and 356b are formed in, respectively, the distal end of the wall 354a and the distal end of the wall 354b. As shown in FIG. 5B, the flange 346 extending radially outwardly from the plunger is received in the groove 356a of the wall 354a when the guard member 332 is in the extended position. As a consequence, the wall 354a directly contacts and prevents the flange 346, which extends outwardly from the plunger, from sliding down the camming surface 348. This, in turn, prevents the biasing member from de-energizing and urging the plunger in the distal direction. The wall 354b may perform a similar retaining function on the opposite side of the injection device. When the guard member 332 is pressed against the patient's skin and retracts into the opening of the housing, the wall 354a may slide out of engagement with the flange 346. This frees the flange 346 such that the flange 346 can slide down the camming surface 358 and into the into the longitudinally extending slot 352 under the biasing force of the biasing member. When the flange 346 is within the longitudinally extending slot 352, the biasing member may continue to de-energize, thereby driving the plunger in the distal direction to expel the drug from the drug storage container.

While each of the foregoing embodiments employs the guard member to release a biasing member of the drive mechanism, the scope of the present disclosure is not limited to this configuration. Alternative embodiments, such as certain of those discussed below, may employ an activator member configured to move independently of the guard member in order to release, activate, and/or unlock the drive mechanism. This activator member may be pressed, along with the guard member, against the patient's skin at the injection site, or, alternatively, the activator member may be actuated by the user with his or her hand, preferably without the user having to change his or her grip of the injection device.

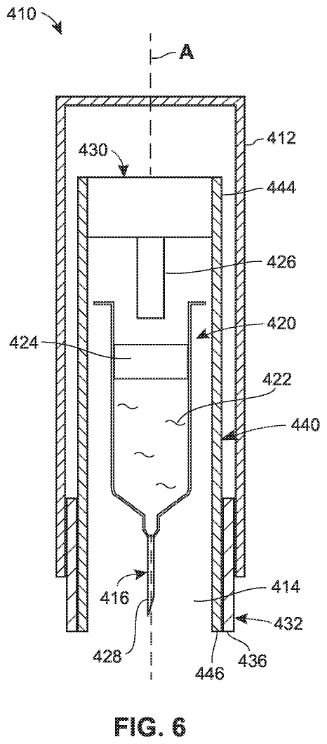

FIG. 6 is a schematic representation of an injection device 410 which is similar to the injection device 10 in FIG. 1, except that the injection device 410 incorporates an activator member 440 that is moveable independent of a guard member 432. Elements in FIG. 6 which are similar in function and/or structure to elements in FIG. 1 are designated by the same reference numeral, incremented by 400 relative to their counterparts in FIG. 1. A description of many of these elements is abbreviated or even eliminated in the interest of conciseness.

Referring to FIG. 6, the activator member 440 may be arranged adjacent to and/or coaxial with the guard member 432 in certain embodiments. The activator member 440 may have a proximal end received within the housing 412, and may be configured to move relative to the housing 412 between an extended position wherein the distal end of the activator member 440 extends through the opening 414 in the housing 412 and a retracted position wherein the distal end of the activator member 440 is retracted, fully or partially, into the opening 414 in the housing 412. In the extended position, the activator member 440 may extend beyond and/or surround the insertion end 428 of the delivery member 416. In some embodiments, moving the activator member 440 toward the retracted position may expose the insertion end 428 of the delivery member 416. In such embodiments, the activator member 440 may serve as a secondary needle guard. In alternative embodiments, the configuration of the activator member 440 may be such that it provides little or no protection against inadvertent needle sticks. In some embodiments, the activator member 440 may be coupled to the housing 412 via, for example, a pin-and-slot arrangement or similar arrangement such that the activator member 440 is able to translate in a linear direction relative to the housing 412 but is prevented from rotating relative to the housing 12.

The activator member 440 is configured to move independently of the guard member 432, at least during retraction of the activator member 440. As such, the guard member 432 does not push or otherwise act on the activator member 440 to cause the activator member 440 to move to the retracted position. The guard member 432 may be configured to move relative to the activator member 440, and vice versa. In some embodiments, the guard member 432 may slide against each other during this relative movement, although this is not necessarily required.

The proximal and distal ends of the activator member 440 may include, respectively, an activator portion 444 and a skin-contacting portion 446. In some embodiments, the activator portion 444 and the skin-contacting portion 446 may be integrally formed to define a single, monolithic structure. Said another way, the activator portion 444 and the skin-contacting portion 446 may be formed in one piece. In other embodiments, the activator portion 444 and the skin-contacting portion 446 may be physically separate structures that are fixedly attached to each other such that they are immovable relative to each other and/or move jointly when in motion. In some embodiments, the activator portion 444 initially may be spaced apart from the skin-contacting portion 446 by a gap, and upon retraction of the skin-contacting portion 446 in the proximal direction, the skin-contacting portion 446 may close the gap and push or otherwise act on the activator portion 444 to move the activator portion 444 relative to the housing 412. At least the skin-contacting portion 446 of the activator member 440 may have a tubular or cylindrical shape and, in some embodiments, may be centered about the longitudinal axis A of the injection device 410. In some embodiments, moving the activator member 440 from the extended position to the retracted position may be accomplished by pressing the skin-contacting portion 446 against the patient's skin at the injection site. In embodiments where the delivery member 416 protrudes from the opening 414 in the housing 412 in the initial or storage state, this motion may result in the insertion end 428 of the delivery member 416 being inserted into the patient's skin.

In some embodiments, the activator member 440 may be biased towards the extended position by a biasing member such as a spring. A user may overcome a biasing force provided by this biasing member by pressing the activator member 440 against, for example, the injection site. When the injection is complete and the injection device 410 is lifted off of the injection site, the biasing member may return the activator member 440 to the extended position, thereby covering the insertion end 428 of the deliver member 416. In some embodiments, the injection device 410 may include a lockout mechanism for locking the activator member 440 in the extended position after the activator member 440 has moved from the retracted position to the extended position in order to prevent re-use of the injection device 410. In some embodiments, only the activator member 440 and not the guard member 320 may return to the extended position after delivery, or vice versa. In still further embodiments, both the activator member 440 and the guard member 320 may return to the extended position after delivery.

The activator member 440 may be configured to interact with the drive mechanism 430 when the activator member 440 moves from the extended position to the retracted position. This interaction may cause the drive mechanism 430 to output the energy necessary for driving the plunger 426 to expel the drug 422 from the drug storage container 420 and/or insert the insertion end 428 of the delivery member 416 into the patient's skin. The interaction between the activator member 440 and the drive mechanism 430 may be achieved by directly coupling the activator member 440 to the drive mechanism 430 or indirectly coupling the activator member 440 to the drive mechanism 430 via, for example, a mechanical or electromechanical linkage. In embodiments where the drive mechanism 430 includes a biasing member such as a spring, movement of the activator member 440 from the extended position to the retracted position may release the biasing member from an energized state to allow the biasing member to drive the plunger 426 to expel the drug 422 from the drug storage container 420. Additionally or alternatively, the activator member 440 may be configured to retain the biasing member in the energized state when the activator member 440 is arranged in the extended position. In some embodiments, the activator member 440 may retain the biasing member via direct contact with biasing member, the plunger 426, and/or an element fixedly attached to the biasing member or plunger 426.

In embodiments where the drive mechanism 430 includes an electromechanical arrangement including an electric motor and/or solenoid and a drive train or transmission coupled to the plunger 426 or an arrangement that generates or releases a pressurized gas or fluid to propel the plunger 426 or which acts directly on the stopper 424, the activator member 440 may directly act on (i.e., directly contact and exert a force on) the drive mechanism 430 to activate the drive mechanism 420 when the activator member 440 moves from the extended position to the retracted position.

In some embodiments, the guard member 332 may not interact with the drive mechanism 430 and actuation of the activator member 440 may be solely responsible for activating the drive mechanism 430. In alternative embodiments, the guard member 332 may play a role in activating the drive mechanism 430. In certain such alternative embodiments, retraction of the guard member 332 may unlock the drive mechanism 430, which may not itself cause the drive mechanism 430 to output the energy necessary for driving the plunger 436, but does allow the activator member 440 to subsequently interact with the drive mechanism 430 so as to cause the drive mechanism 430 to output the energy needed for driving the plunger 436 to expel the drug 422 from the drug storage container 420.

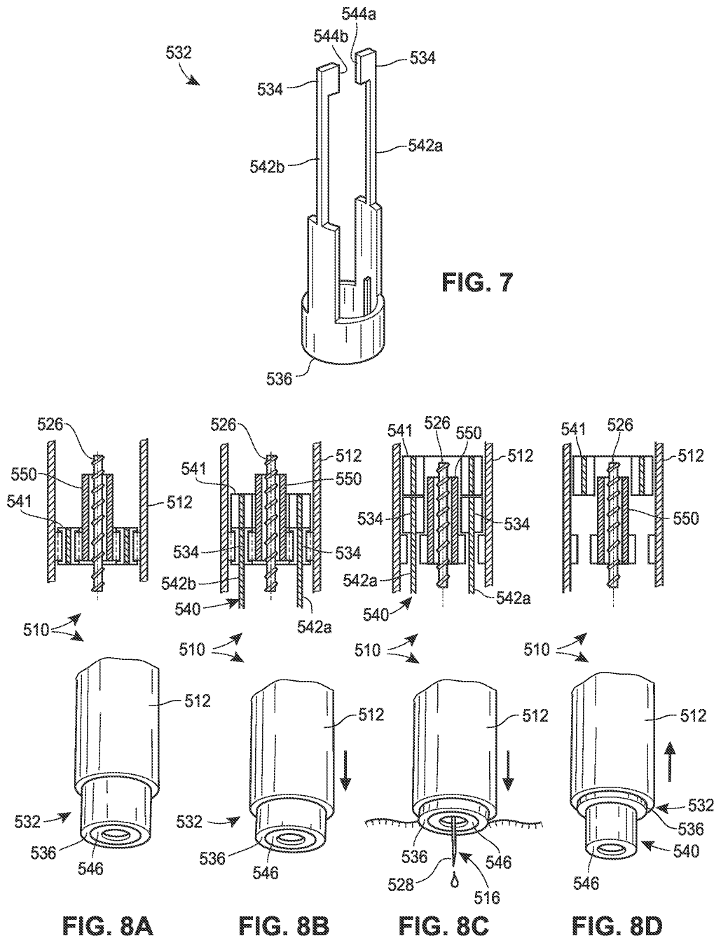

Turning to FIGS. 7-8D, illustrated is an embodiment of an activator member which can be implemented in an injection device including, for example, the injection device illustrated in FIG. 6. Elements in FIGS. 7-8D which are similar in function and/or structure to elements in FIG. 6 are designated by the same reference numeral, incremented by 100 relative to their counterparts in FIG. 6. The activator member 540 here is configured to interact with a retaining member 541, which is a structure that is separate from the activator member 540, as a consequence of the activator member 540 moving from the extended position to the retracted position. This interaction releases a biasing member included in the drive mechanism 430 such that the biasing member is permitted to drive the plunger 526 to expel the drug 422 from the drug storage container 420. In some embodiments, the retaining member 541 may be part of the drive mechanism 30 depicted in FIG. 6.

FIG. 7 shows that the skin-contacting portion 546 of the activator member 540 may have a tubular or cylindrical shape. Two longitudinally extending arms 552a and 552b extend away from the skin-contacting portion 546 in the proximal direction. The skin-contacting portion 546 may be integrally formed with the longitudinally extending arms 552a and 552b to define a single, monolithic structure. Said another way, the skin-contacting portion 546 and the longitudinally extending arms 552a and 552b may be formed in one piece. The longitudinally extending arms 552a and 552b may be parallel or substantially parallel to the longitudinal axis A of the injection device 510 and arranged so as to not cover one or more windows formed in the housing 412 of the injection device 510.

One or both of the proximal ends of the longitudinally extending arms 552a and 552b may define the activator portion 544 of the activator member 540. In the embodiment illustrated in FIG. 7, the proximal ends of the longitudinally extending arms 552a and 552b include, respectively, walls 554a and 554b, each of which extends inwardly away from a remainder of the longitudinally extending arm. The walls 554a and 554b may be perpendicular or substantially perpendicular, or otherwise non-parallel, to the longitudinal axis A of the injection device 510.

When assembled within the injection device, the skin-contacting portion 546 of the activator member 540 may be coaxial with and disposed radially inward of the guard member 530. In the initial state, the skin-contacting portion 536 of the guard member 530 may surround skin-contacting portion 546 of the activator member 540, as shown in FIG. 8A. In alternative embodiments, it may be the skin-contacting portion 546 of the activator member 540 which surrounds the skin-contacting portion 536 of the guard member 530.

Referring to FIGS. 8A-8D, a sequence in which the activator member 540 is used to release a rotational biasing member of the drive mechanism of an injection device, including, e.g., the injection device 410 in FIG. 6, will now be explained. FIG. 8A illustrates an initial state of the injection device 510 prior to activation. Here, the rotational biasing member of the drive mechanism is retained in an energized state by the retaining member 541 arranged in a first position. In the energized state, the rotational biasing member may exert a force biasing a power sleeve 555 to rotate. Initially, however, the power sleeve 555 is prevented by rotating due to an interior surface of the retaining member 541 lockingly engaging an exterior surface of the power sleeve 555. In some embodiments, this interface may include longitudinally extending slot(s) on one of the power sleeve 555 and the retaining member 541 receiving protrusions formed on the other one of the power sleeve 555 and the retaining member 541. FIG. 8A shows that no portion of the activator member 540 is in contact with the retaining member 541 in the initial state; however, in other embodiments, the activator portion 544 of the activator member 540 may be in direct contact with the retaining member 541 in the initial state.

FIG. 8B illustrates a state of the injection device 510 after the skin-contacting portion 546 of the activator member 540 has been pressed against the patient's skin and partially retracted within the opening 514 of the housing 512. Here, the walls 554a and 554b of the activator portion 544 of the activator member 540 directly contact and push the retaining member 541 in the proximal direction. As a consequence, the retaining member 541 is moved from the first position to a second position where the retaining member 541 no longer contacts or restrains rotational movement of the power sleeve 555. As a result of this action, the walls 554a and 554b momentarily assume the first position of the retaining member 541 and thus momentarily retain the rotational biasing member in the energized state by engaging and rotationally locking the power sleeve 555. This may involve the walls 554a and 554b sliding into respective longitudinally extending grooves formed in the exterior surface of the power sleeve 555, which were previously occupied by the inwardly extending protrusions of the retaining member 541.

In the illustrated embodiment, the guard member 530 is pressed against the patient's skin simultaneously with the activator member 540. However, in other embodiments, the guard member 530 may contact the patient's skin prior to the activator member 540, or vice versa.

Upon further retraction of the activator member 540, the walls 554a and 554b may slide out of contact with the power sleeve 555, as shown in FIG. 8C. With nothing to restrain it, the power sleeve 555 may rotate under the rotational biasing force of the rotational biasing member, which may include, for example, a torsion spring (e.g., a helical torsion spring, a spiral torsion spring, etc.). A threaded inner surface of the power sleeve 555 may engage a threaded outer surface of the plunger 526. Consequently, rotation of the power sleeve 555 may drive the plunger 526 in the distal direction to expel the drug from the drug storage container. Once a dose of the drug has been delivered to the patient, the injection device 510 may be lifted off of the patient's skin and the activator member 540 may return to the extended position under the force of a biasing member, as shown in FIG. 8D.

While the embodiment in FIGS. 7-8D utilizes the retaining member 541 for initially retaining the rotational biasing member in the energized state, other embodiments may omit the retaining member 541 and instead utilize the activator member 540 for initially retaining the rotational biasing member in the energized state. For example, in the initial state, when the activator member 540 is in the extended position, the walls 554a and 554b of the activator portion 544 of the activator member 540 may be received within respective grooves formed in the exterior surface of the power sleeve 555 to prevent the power sleeve 555 from rotating. Upon movement of the activator member 540 from the extended position to the retracted position, the walls 554a and 554b may slide out of contact with the power sleeve 555, thereby releasing the power sleeve 555 so that the power sleeve 555 can rotate under the rotational biasing force of the rotational biasing member and thereby threadably advance the plunger 526 in the distal direction to expel the drug 422.

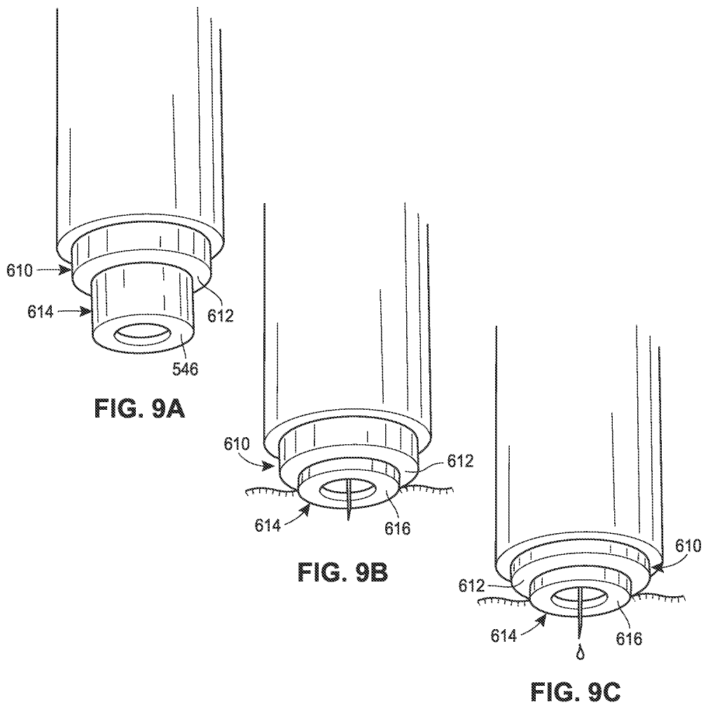

FIGS. 9A-13 depict several variants of an activator member that is moveable independent of the guard member and which can be implemented in any of the injection devices illustrated in FIGS. 6-8D, as well as other injection devices. Also, similar to the embodiments in FIGS. 6-8D, the guard member in the embodiments in FIGS. 9A-13 may not be operably coupled to or otherwise interact with the drive mechanism of the injection device in order to activate, release, and/or unlock the drive mechanism.

FIGS. 9A-9C illustrate an embodiment where the activator member 610 has a skin-contacting portion 612 with a tubular or cylindrical shape and which surrounds a skin-contacting portion 616 of a guard member 614. Furthermore, in the initial state, prior to contact with the patient's skin, the guard member 614 may extend distally beyond the activator member 610, such that the skin-contacting portion 616 of the guard member 614 is exposed (FIG. 9A). As a result, the skin-contacting portion 616 of the guard member 614 makes contact with the patient's skin at the injection site prior to the skin-contacting portion 612 of the activator member 610. Upon skin contact, initially the guard member 614 retracts into the housing of the injection device while the activator member 610 remains stationary relative to the housing of the injection device. Once the skin-contacting portion 616 of the guard member 614 retracts to a position where it is even with the skin-contacting portion 612 of the activator member 610 (FIG. 9B), the skin-contacting portion 612 of the activator member 610 makes contact with the patient's skin and begins to retract into the housing. Subsequently, both the activator member 610 and the guard member 614 are pushed to their respective retracted positions (FIG. 9C). As described above in connection with FIGS. 6-8D, retraction of the activator member 610 may cause the activator member 610 to interact, directly or indirectly, with a drive mechanism in order to release, activate, and/or unlock the drive mechanism, which, in turn, causes the drive mechanism to output energy for driving a plunger to expel a drug from a drug storage container into the patient and/or inserting the insertion end of the delivery member into the patient's skin. In some embodiments, after drug delivery is complete and the injection device is lifted off of the patient's skin, the activator member 610 may be locked in its retracted position while the guard member 614 is deployed back to its extended position for needle stick prevention purposes.

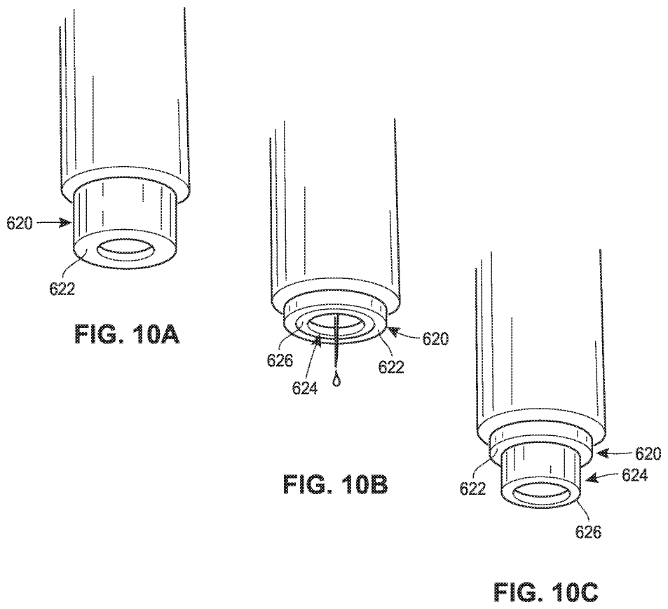

FIGS. 10A-10C illustrate an embodiment similar to the embodiment in FIGS. 9A-9C, except that in the initial state the activator member 620 extends distally beyond the guard member 624, such that a skin-contacting portion 626 of the guard member 624 is covered by a skin-contacting portion 622 of the activator member 620 (FIG. 10A). As a consequence, the skin-contacting portion 622 of the activator member 620 makes contact with the patient's skin at the injection site prior to the skin-contacting portion 626 of the guard member 624. Upon skin contact, initially the activator member 620 retracts into the housing of the injection device while the guard member 624 remains stationary relative to the housing of the injection device. Once the skin-contacting portion 622 of the activator member 620 retracts to a position where it is even with the skin-contacting portion 626 of the guard member 624, the skin-contacting portion 626 of the guard member 624 makes contact with the patient's skin and begins to retract into the housing, along with the activator member 620. Subsequently, both the activator member 610 and the guard member 614 are pushed to their respective retracted positions (FIG. 10B). As described above in connection with FIGS. 6-8D, retraction of the activator member 620 may cause the activator member 620 to interact, directly or indirectly, with a drive mechanism in order to release, activate, and/or unlock the drive mechanism, which, in turn, causes the drive mechanism to output energy for driving a plunger to expel a drug from a drug storage container into the patient and/or inserting the insertion end of the delivery member into the patient's skin. After drug delivery is complete and the injection device is lifted off of the patient's skin, the activator member 620 may be locked in its retracted position while the guard member 624 is deployed back to its extended position (FIG. 10C). Thus, after the injection, the guard member 624 but not the activator member 620 provides protection against inadvertent needle sticks. In a variant of the embodiment in FIGS. 10A-10C, the guard member 624 may initially be retained in a retracted position and not be deployed to the extended position until after drug delivery device. In such alternative embodiments, the guard member 624 may not make contact with the patient's skin at the injection site during retraction of the activator member 620.

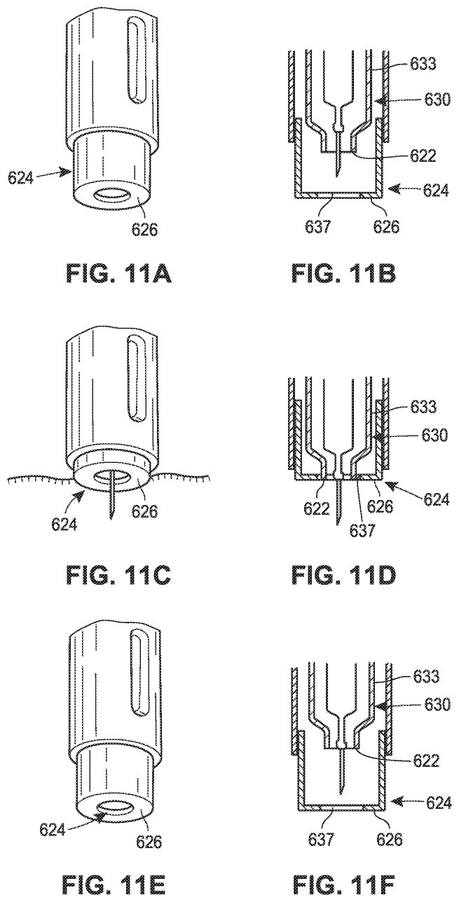

FIGS. 11A-11F illustrate an embodiment similar to the embodiment in FIGS. 9A-9C, except that the activator member 630 is surrounded by the guard member 634 at all times. Here, a skin-contacting portion 632 of the activator member 630 may have a smaller diameter or width than an activator portion 633 of the activator member 630. Furthermore, the diameter of the skin-contacting portion 632 may be smaller than an opening 637 in the skin-contacting portion 636 of the guard member 634. So configured, the skin-contacting portion 632 of the activator member 630 may fit through the opening 637 and come into contact with the patient's skin upon retraction of the guard member 634. This may push the activator member 630 to the retracted position shown in FIGS. 11C and 11D. The larger diameter or width of the activator portion 633 of the activator member 630 may allow the activator portion 633 to accommodate the barrel of the drug storage container. After drug delivery is complete and the injection device is lifted off of the patient's skin, the activator member 630 may be locked in its retracted position while the guard member 634 is deployed back to its extended position (FIGS. 11F and 11F).

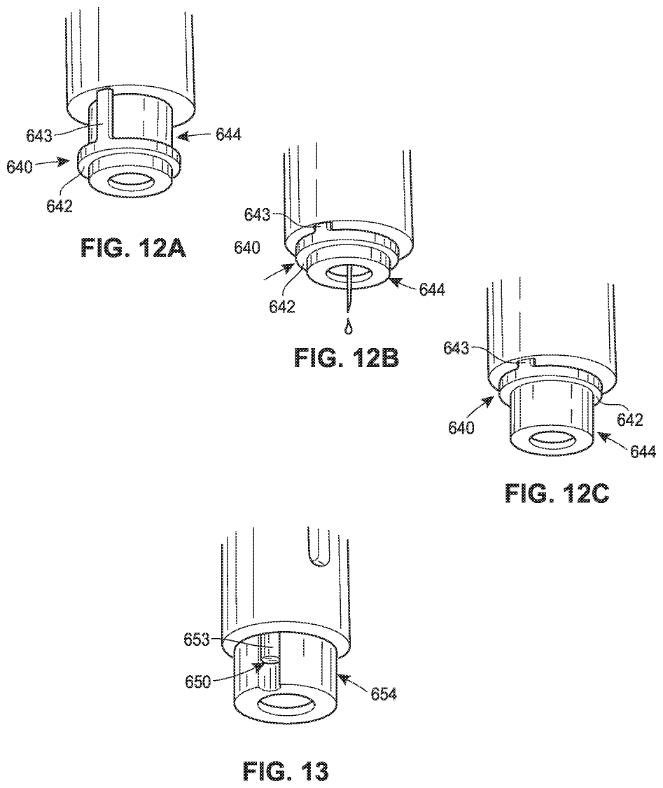

FIGS. 12A-12C depict an embodiment similar to the embodiment in FIGS. 9A-9C, except that the activator member 640 has a different shape. Similar to its counterpart in the embodiment in FIGS. 9A-9C, the distal end of the activator member 640 is disposed around the guard member 644 and has a generally tubular or cylindrical shape. Different from its counterpart in the foregoing embodiment, the tubular portion of the activator member 640, which may be considered to be a ring, does not span the entire distance between the housing of injection device and the skin-contacting portion 642 of the activator member 640. As a consequence, in the initial state in FIG. 12A, a portion of the guard member 644 located axially between the housing and the skin-contacting portion 642 of the activator member 640 is not covered by the activator member 640. A longitudinally extending arm or rod 643, which may define the activation portion of the activator member 640, extends alongside the guard member 644 in a direction away from the skin-contacting portion 642 of the activator member 640. As seen in FIG. 12A, a portion of the rod 643 is disposed outside of the opening in the housing of the injection device in the initial state. In a similar manner as its counterpart in the embodiment in FIGS. 9A-9C, the activator member 640 releases, activates, and/or unlocks the drive mechanism of the injection device when the activator member 640 moves from its extended position (FIG. 12A) to its retracted position (FIG. 12B). After drug delivery is complete and the injection device is lifted off of the patient's skin, the activator member 640 may be locked in its retracted position while the guard member 644 is deployed back to its extended position (FIG. 12C).

FIG. 13 illustrates a variant of the embodiment in FIGS. 12A-12C. Here, the activator member 650 lacks a tubular portion and the longitudinally extending arm or rod 653 defines both the skin-contacting portion of the activator member 650 and the activation portion of the activator member 650. FIG. 13 shows that the rod 653 is disposed within a groove or slot in the wall of the guard member 654; however, in alternative embodiments, the rod 653 may be arranged alongside the guard member 654, either radially inside or radially outside of the guard member 654.

In any of the embodiments described in connection with FIGS. 6-13, the injection device may include one or more mechanisms for positioning the activator member and the guard member according to any of the following schemes. According to one scheme, in the initial state (e.g., prior to disposal of the injection device against the patient's skin at the injection site), the guard member may be retained or otherwise arranged in the retracted position and the activator member may be biased to or otherwise arranged in the extended position. Later, in a post-delivery state (e.g., after the drug has been delivered to the patient and the injection device has been removed from the injection site), the guard member may be automatically deployed to the extended position and the activator member may be retained in the retracted position or automatically deployed to the extended position. According to another scheme, in the initial state, the guard member may the activator member may be retained or otherwise arranged in the retracted position and the guard member may be biased to or otherwise arranged in the extended position. Later, in the post-delivery state, the activator member may be automatically deployed to the extended position and the guard member may be retained in the retracted position or automatically deployed to the extended position.

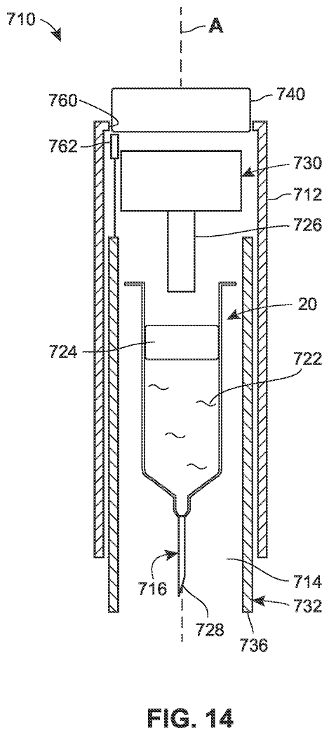

While the embodiments of the activator member described in connection with FIGS. 6-13 require the activator member to extend through the same opening in the housing as the guard member, alternative embodiments can be arranged differently. FIG. 14 illustrates an embodiment of an activator member disposed at a proximal end of the housing of the injection device and which does not make contact with the patient's skin at the injection site. Rather, the activator member is manually operable by user with his or her hand while the distal end of the injection device is pressed against the patient's skin at the injection site.

More particularly with respect to FIG. 14, elements which are similar in function and/or structure to elements in FIG. 6 are designated by the same reference numeral, incremented by 300 relative to their counterparts in FIG. 6. A description of many of these elements is abbreviated or even eliminated in the interest of conciseness. FIG. 14 illustrates an activator member 740 slidably received within an opening 760 formed in a proximal end of the housing 712. The activator member 740, unless restricted by the lock mentioned below, is moveable relative to the housing 712 through the opening 760. The activator member 740 is operably coupled to the drive mechanism 730 such that movement of the activator member 740 relative to the housing 712 causes the activator member 740 to interact, directly or indirectly, with the drive mechanism 730 in order to release, activate, and/or unlock the drive mechanism 730, which, in turn, causes the drive mechanism 730 to output energy for driving the plunger 726 to expel the drug 722 from the drug storage container 720 and/or inserting the insertion end 728 of the delivery member 716 into the patient's skin.

As shown in FIG. 14, the activator member 740 may take the form of a push button sized and dimensioned for manipulation by a users thumb or other finger. A user may grip the circumferential surface of the housing 712 in the palm of their hand, and, without having to change their grip, use their thumb to actuate the activator member 740. In the illustrated embodiment, the activator member 740 may be configured to move in a direction parallel or substantially parallel to the longitudinal axis A of the injection device 710. In other embodiments, the activator member 740 may be configured to move in a direction perpendicular, substantially perpendicular, or otherwise non-parallel to the longitudinal axis A of the injection device 710. In some embodiments, movement of the activator member 740 may follow an arcuate path, including, e.g., one centered about the longitudinal axis A.

In some embodiments, the injection device 710 may include a lock 762 operably coupled to the activator member 740 and configured to selectively permit movement of the activator member 740 relative to the housing 712. The lock 762 may have a locked state wherein the lock 762 prevents movement of the activator member 740 and an unlocked state wherein the lock 762 permits movement of the activator member 740. Furthermore, the lock 762 may be operably coupled to the guard member 732 such that moving the guard member 732 from the extended position to retracted position when pressing the guard member 732 against the injection site causes the lock 762 to change from the locked state to the unlocked state. Accordingly, the activator member 740 is able to move and thus activate, release, and/or unlock the drive mechanism 730 only when the guard member 732 has moved from the extended position to the retracted position. This helps reduce the likelihood of premature activation of the drive mechanism 730, which may result in open air discharge of the drug 722. In alternative embodiments, the lock 762 may be omitted. In such embodiments, the activator member 740 may be free to move and activate, release, and/or unlock the drive mechanism 730 independent of the position of the guard member 732.

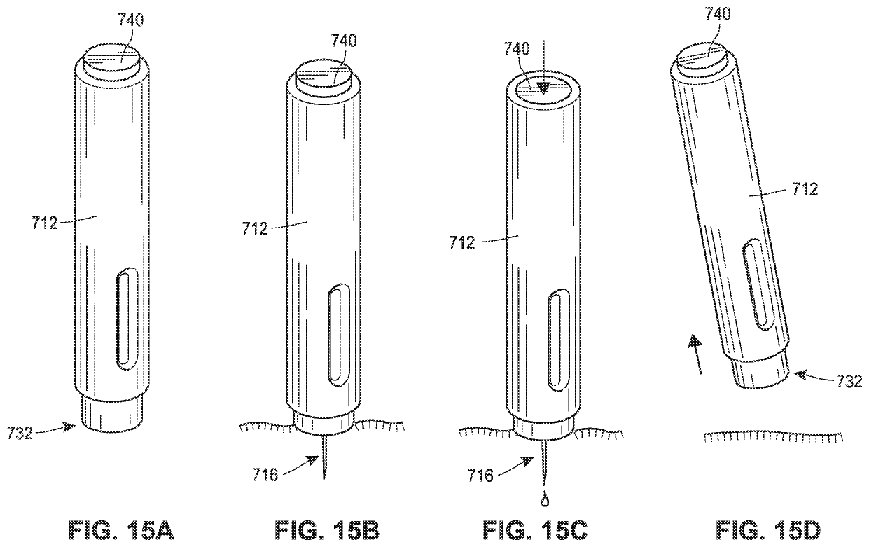

FIGS. 15A-15D illustrate a sequence in which the injection device 710 is used to perform an injection. FIG. 15A illustrates an initial state prior to activation. Here, the guard member 732 is biased to its extended position and the lock 762 is in the locked state. As such, the lock 762 prevents movement of the activator member 740 relative to the housing 712 in this state. Subsequently, in FIG. 15B, the guard member 732 is pressed against the patient's skin at the injection site, causing the guard member 732 to move from the extended position to the retracted position. This movement causes the guard member 732 to interact with the lock 762, changing the lock 762 from the locked state to the unlocked state. Next, the user may push the activator member 740 with his or her thumb in the distal direction into the opening 760 in the housing 712 (FIG. 15C). As a consequence, the activator member 740 interacts with the drive mechanism 730 in order to release, activate, and/or unlock the drive mechanism 730, which, in turn, causes the drive mechanism 730 to output energy for driving the plunger 726 to expel the drug 722 from the drug storage container 720 and, if the insertion end 728 of the delivery member 716 was not previously inserted into the patient during retraction of the guard member 732, inserting the insertion end 728 of the delivery member 716 into the patient's skin. Once a dose of the drug 722 has been delivered to the patient, the injection device 710 may be lifted off of the patient's skin and the guard member 732 may return to the extended position under the force of a needle guard biasing member, as shown in FIG. 15D.

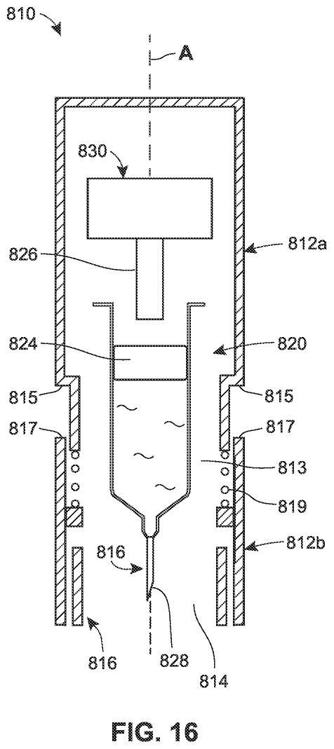

In each of the foregoing embodiments, the activator member is a structure that is separate from the housing of the injection device. Alternative embodiments, such as the one in FIG. 16, may be configured such that the housing, or a portion thereof, functions as the activator member. The injection device in FIG. 16 includes many elements which are structurally and/or functionally similar to elements of injection device in FIG. 14. Such elements are identified with the same reference numeral, incremented by 100 relative to their counterparts in FIG. 14.

FIG. 16 illustrates that the injection device 810 has a proximal housing 812a and a distal housing 812b. The proximal and distal housings 812a and 812b may each have a generally tubular or cylindrical shape and may be centered about the longitudinal axis A of the injection device 810. The proximal housing 812a may be sized and dimensioned such that it can be gripped by a user in the palm of their hand. As such, the proximal housing 812a may define a tubular hand grip. The drive mechanism 830 may be disposed entirely or partially within an interior space of the proximal housing 812a. A distal end of the proximal housing 812a, which may have a smaller diameter or width than a proximal end of the proximal housing 812a, may be slidably received in an opening 813 formed in the proximal end of the distal housing 812b. The proximal housing 812a is moveable relative to the distal housing 812b, with the distal end of the proximal housing 812a being inserted into the opening 813 when the proximal housing 812a moves in the distal direction. Furthermore, the proximal housing 812a is operably coupled to the drive mechanism 830 such that movement of the proximal housing 812a relative to the distal housing 812b in the distal direction causes the proximal housing 812a to interact, directly or indirectly, with the drive mechanism 830 in order to release, activate, and/or unlock the drive mechanism 830, which, in turn, causes the drive mechanism 830 to output energy for driving the plunger 826 to expel the drug 822 from the drug storage container 820 and/or inserting the insertion end 828 of the delivery member 816 into the patient's skin.

In an initial state, as depicted in FIG. 16, a distally facing surface 815 of the proximal housing 812a may be spaced by an axial distance or gap from a proximally facing surface 817 of the distal housing 812b. A biasing member 819 such as a spring may be arranged between the proximal housing 812a and the distal housing 812b and may be configured to exert a biasing force resisting closure of the gap between the distally facing surface 815 of the proximal housing 812a and the proximally facing surface 817 of the distal housing 812b. Additionally, in the initial state, the guard member 832 may be arranged in the retracted position, as shown in FIG. 16.

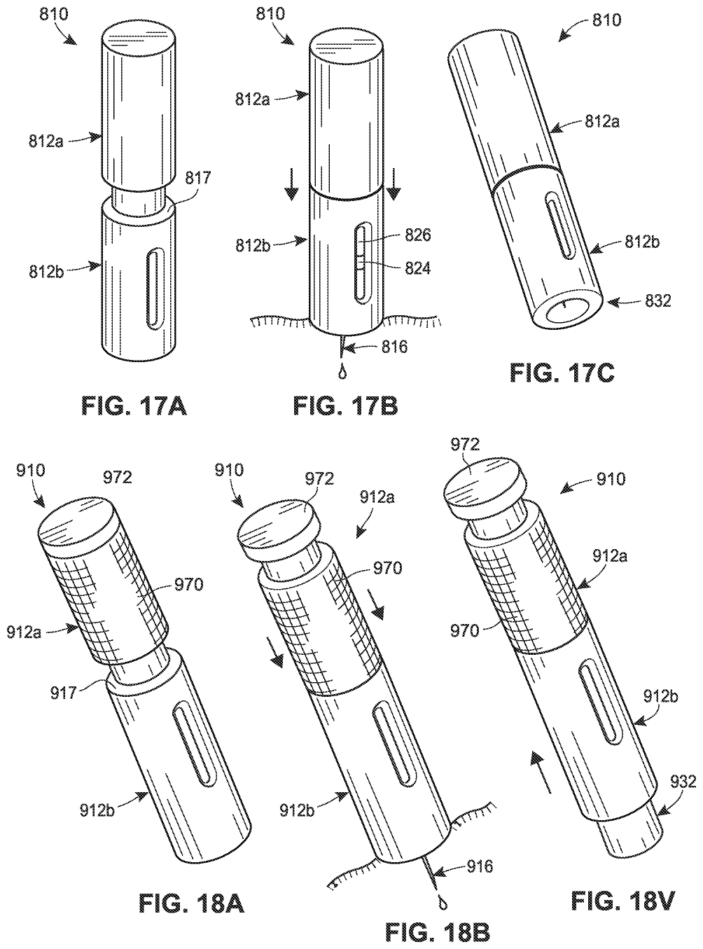

Referring now to FIGS. 17A-17C, a sequence is illustrated in which the injection device 810 is used to perform an injection. FIG. 17A illustrates an initial state of the injection device 810 prior to activation. Here, with no external forces pushing the proximal housing 812a and the distal housing 812b toward each other, the biasing force of the biasing member 819 maintains the gap between the between the distally facing surface 815 of the proximal housing 812a and the proximally facing surface 817 of the distal housing 812b. Subsequently, a user may grip the proximal housing 812a in his or her hand and press the distal housing 812b against the patient's skin at the injection site (FIG. 17B). The manually applied force overcomes the biasing force of the biasing member 819 and the proximal housing 812a moves in the distal direction along the longitudinal axis A toward the distal housing 812b until the distally facing surface 815 of the proximal housing 812a abuts against the proximally facing surface 817 of the distal housing 812b. This distal movement of the proximal housing 812a causes the proximal housing 812a to interact with the drive mechanism and, as a consequence, activates, releases, and/or unlocks the drive mechanism, which, in turn, causes the drive mechanism to output energy that drives the delivery member 816 in the distal direction such that the insertion end 828 of the delivery member 816 pierces the patient's skin and drives the plunger 826 in the distal direction to expel the drug from the drug storage container 820 through the delivery member 816 into the patient. Once a dose of the drug has been delivered to the patient, the injection device 810 may be lifted off of the patient's skin and a biasing member may be released to move the guard member 832 from the retracted position to extended position to cover the insertion end 828 of the delivery member 816 (FIG. 17C).

FIGS. 18A-18C illustrate a variant of the embodiment in FIGS. 16-17C. Here, the proximal housing 912a is divided into two separate structures: an actuating sleeve 970 and an end cap 972. The end cap 972 may be fixed relative to the distal housing 912b such that the end cap 972 does not move relative to the distal housing 912b. The actuating sleeve 970 may have a proximal opening slidably receiving a distal end of the end cap 927 and a distal opening slidably receiving a proximal end of the distal housing 912b. Furthermore, the actuating sleeve 970 may be positioned axially between and moveable relative to the end cap 972 and the distal housing 912b. The actuating sleeve 970 may be operably coupled to the drive mechanism such that movement of the actuating sleeve 970 in the distal direction causes the actuating sleeve 970 to interact, directly or indirectly, with the drive mechanism in order to release, activate, and/or unlock the drive mechanism, which, in turn, causes the drive mechanism to output energy for driving the plunger to expel the drug from the drug storage container and/or inserting the insertion end of the delivery member into the patient's skin.

FIG. 18A illustrates an initial state of the injection device 910 prior to activation. Here, the guard member 932 is in a retracted state and the actuating sleeve 970 is biased, e.g., via the biasing member 819, in the proximal direction such that a proximally facing end surface of the actuating sleeve 970 abuts against a distally facing surface of the end cap 972. In this arrangement, the biasing force of the biasing member maintains a gap between the distally facing end surface of the actuating sleeve 970 and the proximally facing surface of the distal housing 912b. Subsequently, a user may grip the actuating sleeve 970 in his or her hand and press the distal housing 912b against the patient's skin at the injection site (FIG. 18B). The manually applied force overcomes the biasing force and the actuating sleeve 970 moves in the distal direction along the longitudinal axis A toward the distal housing 912b (i.e., away from the end cap 972) until the distally facing end surface of the actuating sleeve 970 abuts against the proximally facing surface of the distal housing 912b. This distal movement of the actuating sleeve 970 causes the actuating sleeve 970 to interact with the drive mechanism and, as a consequence, activates, releases, and/or unlocks the drive mechanism, which, in turn, causes the drive mechanism to output energy that drives the delivery member 916 in the distal direction such that the insertion end 928 of the delivery member 916 pierces the patient's skin and drives the plunger in the distal direction to expel the drug from the drug storage container through the delivery member 916 into the patient. After a dose of the drug has been delivered to the patient, the injection device 910 may be lifted off of the patient's skin and a biasing member may be released to move the guard member 932 from the retracted position to extended position to cover the insertion end 928 of the deliver member 916 (FIG. 18C).

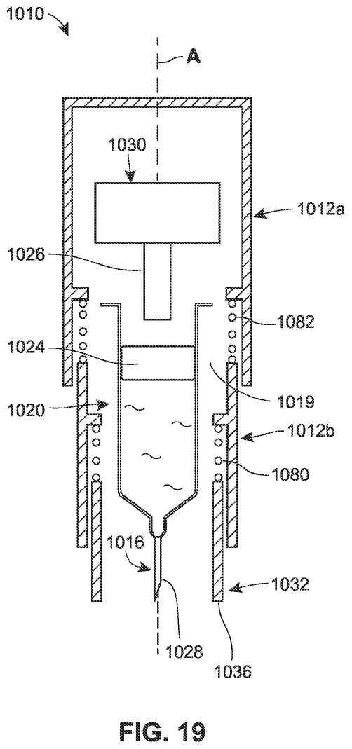

FIG. 19 illustrates an embodiment of an injection device that similar in certain respects to the injection device in FIG. 16, except that the guard member is arranged in the extended position in the initial state, among other differences. The injection device in FIG. 19 includes many elements which are structurally and/or functionally similar to elements of injection device in FIG. 16. Such elements are identified with the same reference numeral, incremented by 200 relative to their counterparts in FIG. 16.

FIG. 19 shows that the injection device 1010 has a proximal housing 1012a and a distal housing 1012b. The proximal and distal housings 1012a and 1012b may each have a generally tubular or cylindrical shape and may be centered about the longitudinal axis A of the injection device 1010. The proximal housing 1012a may be sized and dimensioned such that it can be gripped by a user in the palm of their hand. As such, the proximal housing 1012a may define a tubular hand grip. The drive mechanism 1030 may be disposed entirely or partially within an interior space of the proximal housing 1012a. An opening 1019 may be formed in the distal end of the proximal housing 1012a and may be slidably received a proximal end of the distal housing 1012b. The proximal housing 1012a may moveable relative to the distal housing 1012b, with the proximal end of the distal housing 1012b being inserted into the opening 1019 when the proximal housing 1012a moves in the distal direction. Furthermore, the proximal housing 1012a may be operably coupled to the drive mechanism 1030 such that movement of the proximal housing 1012a relative to the distal housing 1012b in the distal direction causes the proximal housing 1012a to interact, directly or indirectly, with the drive mechanism 1030 in order to release, activate, and/or unlock the drive mechanism 1030, which, in turn, causes the drive mechanism 1030 to output energy for driving the plunger 1026 to expel the drug 1022 from the drug storage container 1020 and/or inserting the insertion end 1028 of the delivery member 1016 into the patient's skin.

The injection device 1010 may include a first biasing member 1080 arranged between the distal housing 1012b and the guard member 1032, and a second biasing member 1082 arranged between the proximal housing 1012a and the distal housing 1012b. In some embodiments, the first and second biasing members 1080 and 1082 may each include a respective spring such as, for example, a compression spring. As shown in FIG. 19, these springs may be operably arranged in series with each other in certain embodiments. The first biasing member 1080 may be configured to exert a first biasing force urging the guard member 1032 toward the extended position. The second biasing member 1082 may be configured to exert a second biasing force urging the proximal housing away from the distal housing. Furthermore, the second biasing force may be greater than the first biasing force. In embodiments where the first and second biasing members 1080 and 1082 are springs, this may be achieved by the second biasing member 1082 having a spring constant which is greater than a spring constant of the first biasing member 1080. So configured, when the guard member 1032 is pressed against patient's skin at the injection site, the guard member 1032 initially moves from the extended position to the retracted position, and subsequently, the proximal housing 1012a moves toward the distal housing 1012b. This sequence may ensure that the insertion end 1028 of the delivery member 1016 is inserted into the patient prior to the activation of the drive mechanism 1030 to expel the drug 1022 from the drug storage container 1020.

In additional to or as an alternative to the series arrangement of the first and second biasing members 1080 and 1082, some embodiments may incorporate a lock operably coupled to the proximal housing 1012a and configured to selectively permit movement of the proximal housing 1012a relative to the distal housing 1012b. This lock may be similar in certain respects to the one discussed above in connection with FIG. 14. The lock may have a locked state wherein the lock prevents movement of the proximal housing 1012a relative to the distal housing 1012b, and an unlocked state wherein the lock permits movement of the proximal housing 1012a relative to the distal housing 1012b. Furthermore, the lock may be operably coupled to the guard member 1032 such that moving the guard member 1032 from the extended position to retracted position when pressing the guard member 1032 against the injection site causes the lock to change from the locked state to the unlocked state. As a result, the proximal housing 1012a is able to move relative to the distal housing 1012b in the distal direction and thereby activate, release, and/or unlock the drive mechanism 1030 only when the guard member 1032 has moved from the extended position to the retracted position.

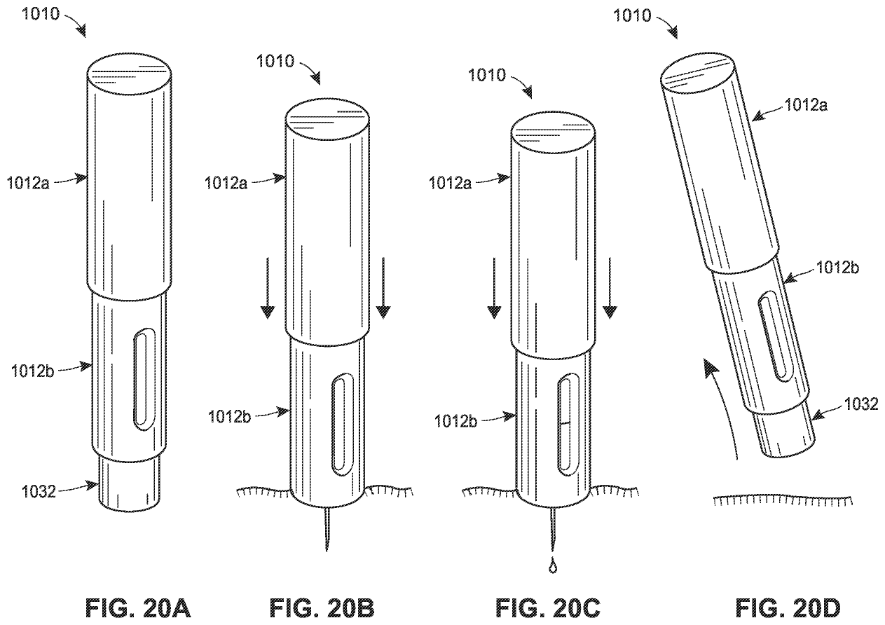

Turning to FIGS. 20A-20D, a sequence is illustrated in which the injection device 1010 is used to perform an injection. FIG. 20A depicts an initial state of the injection device 1010 prior to activation. Here, with no external force(s) urging the guard member 1032 and the proximal housing 1012a toward each other, the guard member 1032 is arranged in the extended position and is biased towards the extended position by the first biasing member 1080. Also, in the initial state, the proximal housing 1012a is arranged in a first position relative to the distal housing 1012b and is biased towards this first position by the second biasing member 1082. In use, a user may grip the proximal housing 1012a in his or her hand and press guard member 1032 against the patient's skin at the injection site (FIG. 20B). The manually applied force overcomes the first biasing force of the first biasing member 1080 and the guard member 1032 moves in the proximal direction until it reaches its retracted position. The insertion end 1028 of the deliver member 1016 may be inserted into the patient's skin as a result. As shown in FIG. 20B, the retracted position of the guard member 1032 may correspond to the guard member 1032 fully retracting into the distal housing 1012b, but this does not necessarily have to occur, so long as the guard member 1032 is retracted to a position where it can no longer retract any further relative to the distal housing 1012b. The user then continues to apply force in the distal direction to overcome the second biasing force of the second biasing member 1082, such that the proximal housing 1012a moves in the distal direction along the longitudinal axis A toward the distal housing 1012b (FIG. 20C). The force needed to cause this movement will be greater than a minimum force needed to retract the needle guard 1032. The distal movement of the proximal housing 1012a causes the proximal housing 1012a to interact with the drive mechanism 1030 and, as a consequence, activates, releases, and/or unlocks the drive mechanism 1030, which, in turn, causes the drive mechanism 1030 to output energy that drives the plunger in the distal direction to expel the drug from the drug storage container through the delivery member into the patient. Once a dose of the drug 1022 has been delivered to the patient, the injection device 1010 may be lifted off of the patient's skin and thed first biasing member 1080 may move the guard member 1032 from the retracted position to extended position to cover the insertion end 1028 of the delivery member 1016 (FIG. 20D).

As will be recognized, the devices and methods according to the present disclosure may have one or more advantages relative to conventional technology, any one or more of which may be present in a particular embodiment in accordance with the features of the present disclosure included in that embodiment. Other advantages not specifically listed herein may also be recognized as well.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like;Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNa mAb (MEDI-545, MDX-1103); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/FIt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. An injection device comprising:
a housing having an opening;
a drug storage container including a delivery member having an insertion end configured to extend at least partially through the opening in the housing;
a plunger;
a biasing member operably coupled to the plunger and initially retained in an energized state, wherein releasing the biasing member drives the plunger to expel a drug from the drug storage container through the delivery member; and
a guard member having a skin-contacting portion and an activator portion, the guard member being moveable relative to the housing and having an extended position wherein the guard member extends at least partially through the opening in the housing and a retracted position wherein the guard member is positioned away from the extended position toward the housing,
wherein moving the guard member from the extended position to the retracted position causes the activator portion to release the biasing member to allow the biasing member to drive the plunger to expel the drug from the drug storage container.

2. The injection device of claim 1, comprising a retaining member having a first position wherein the retaining member retains the biasing member in the energized state and a second position wherein the retaining member is freed from retaining the biasing member, wherein the activator portion acts on the retaining member to move the retaining member from the first position to the second position when the guard member moves from the extended position to the retracted position.

3. The injection device of claim 2, wherein:
(a) the activator portion directly contacts the retaining member to move the retaining member from the first position to the second position, and/or
(b) the guard member having a partially retracted position between the retracted position and the extended position, the activator portion being configured to retain the biasing member in the energized state when the activator portion is in the partially retracted position.

4. (canceled)

5. The injection device of claim 1, the activator portion being configured to retain the biasing member in the energized state when the guard member is in the extended position, and optionally comprising a releaser member configured to rotate under a biasing force exerted by the biasing member, the activator portion being configured to resist rotation of the releaser member when the guard member is in the extended position.

6. (canceled)

7. The injection device of claim 6, wherein:
(a) the activator portion directly contacts the releaser member when the guard member is in the extended position and wherein the activator portion is spaced from the releaser member when the guard member is in the retracted position, and/or
(b) the releaser member being fixed to or integrally formed with the plunger.

8. (canceled)

9. The injection device of claim 1, the skin-contacting portion and the activator portion:
(a) jointly translating in a linear direction between the extended position and the retracted position, and/or
(b) being integrally formed to define a single, monolithic structure.

10. (canceled)

11. The injection device of claim 1, the guard member including a tubular portion and at least one longitudinally extending arm extending away from the tubular portion, an end surface of the tubular portion defining the skin-contacting portion, the activator portion optionally being defined at least in part by a wall extending inwardly from the at least one longitudinally extending arm.

12-14. (canceled)

15. An injection device comprising:
a housing having an opening;
a drug storage container including a delivery member having an insertion end configured to extend at least partially through the opening in the housing;
a plunger;
a drive mechanism activatable to expel a drug from the drug storage container through the delivery member;
a guard member moveable relative to the housing and having a guard member extended position wherein the guard member extends at least partially through the opening in the housing and a guard member retracted position wherein the guard member is positioned away from the guard member extended position toward the housing; and
an activator member moveable relative to the housing independent of movement of the guard member.

16. The injection device of claim 15, wherein the activator member is moveable relative to the housing and has an activator member extended position wherein the activator member extends through the opening in the housing and an activator member retracted position wherein the activator member is positioned away from the activator member extended position toward the housing.

17. The injection device of claim 16, comprising:
the drive mechanism including a rotational biasing member; and
a retaining member having a first position wherein the retaining member retains the rotational biasing member in an energized state and a second position wherein the retaining member releases the rotational biasing member to allow the rotational biasing member to rotate, wherein the activator member acts on the retaining member to move the retaining member from the first position to the second position when the activator member moves from the activator member extended position to the activator member retracted position.

18. The injection device of claim 16, the activator member being operably coupled to the drive mechanism such that moving the activator member from the activator member extended position to the activator member retracted position activates the drive mechanism.

19. The injection device of claim 18, wherein the activator member, in moving from the activator member extended position to the activator member retracted position:
(a) directly contacts and interacts with the drive mechanism to activate the drive mechanism, or
(b) releases a biasing member of the drive mechanism.

20. (canceled)

21. The injection device of claim 16, wherein the guard member in the guard member extended position extends beyond the activator member in the activator member extended position.

22. The injection device of claim 15, wherein:
(a) the activator member includes a skin-contacting portion and surrounds at least a portion of the guard member, and/or
(b) the guard member surrounding at least a portion of the activator member, and the activator member having a skin-contacting portion.

23. (canceled)

24. The injection device of claim 16, the guard member includes a tubular skin-contacting portion and the activator member includes a longitudinally extending arm disposed through the opening in the housing when the activator member is in the activator member extended position

25-27. (canceled)

28. The injection device of claim 16, wherein, in an initial state, the guard member is in the guard member retracted position and the activator member is in the activator member extended position, and optionally, in a post-delivery state, the guard member is in the guard member extended position and the activator member is in the activator member extended position or the activator member retracted position.

29. (canceled)

30. The injection device of claim 16, wherein, in an initial state, the activator member is in the activator member retracted position and the guard member is in the guard member extended position, and optionally, in a post-delivery state, the activator member is in the activator member extended position and the guard member is in the guard member retracted position or the guard member extended position.

31-32. (canceled)

33. An injection device comprising:
a distal housing having an opening;
a drug storage container disposed at least partially in the distal housing and including a delivery member having an insertion end configured to extend at least partially through the opening in the distal housing;
a plunger;
a drive mechanism activatable to drive the plunger in a distal direction to expel a drug from the drug storage container through the delivery member; and
a proximal housing operably coupled to the drive mechanism and moveable relative to the distal housing such that moving the proximal housing in the distal direction activates the drive mechanism.

34. The injection device of claim 33, comprising a guard member moveable relative to the distal housing and having an extended position wherein the guard member extends at least partially through the opening in the distal housing and a retracted position wherein the guard member is positioned away from the extended position toward the distal housing.

35. The injection device of claim 33, wherein:
(a) the proximal housing being cylindrical and sized and dimensioned to be gripped in a hand of a user,
(b) at least a portion of the drive mechanism is disposed in the proximal housing,
(c) the proximal housing having an initial position wherein a distally facing surface of the proximal housing is spaced from a proximally facing surface of the distal housing, and an activation position wherein the distally facing surface of the proximal housing abuts against the proximally facing surface of the distal housing,
(d) a distal end of the proximal housing is received in a second opening in a proximal end of the distal housing, and/or
(e) a proximal end of the distal housing is received in an opening in a distal end of the proximal housing.

36-46. (canceled)
